(19) United States
(12) Patent Application Publication
(10) Pub. No.: US 2006/0217683 A1
(43) Pub. Date: Sep. 28, 2006

(54) Luer connectors and fitting

(76) Inventor: Andrew C Patania, Whitmore Lake, MI (US)

Correspondence Address: LEYDIG VOIT & MAYER, LTD, 700 THIRTEENTH ST. NW, SUITE 300, WASHINGTON, DC, 20005-3960, US

(21) Appl. No.: 10/540,360

(22) PCT Filed: Jan. 15, 2004
(86) PCT No.: PCT/US04/00854

§ 371 Date: May 18, 2006

Related U.S. Application Data
(60) Provisional application No. 60/440,031, filed on Jan. 15, 2003.

(51) Int. Cl. A61M 39/10

(52) U.S. Cl. 604/533

(57) ABSTRACT

A luer connector may comprise a female fitting and a male fitting. The female fitting may have an open end including a bore and an opening at the end of the bore. The bore has a taper and the opening has a diameter which are specified in accordance with ISO International Standard 594. The male fitting may have a projection which is capable of fitting within the tapered bore of the female fitting. The male fitting may also include a fluid passage which extends through the projection and has an effective inner diameter of at least about 0.130 inch.

This application claims the benefit of U.S. Provisional Application No. 60/440,031, which is incorporated herein by reference.

### FIELD OF THE INVENTION

This invention relates to luer connectors and fittings. More particularly, it relates to luer connectors and fittings that provide reduced resistance to fluid flow.

### BACKGROUND OF THE INVENTION

Luer connectors provide reliable connections for a wide variety of devices or assemblies through which a fluid, such as a gas, a liquid or a mixture of gases, liquids and/or solids, flows. For example, luer connectors may be used with tubing, syringes, needles, infusion sets, insufflation devices, transfusion sets, and many other assemblies, often designed for medical and pharmaceutical applications, as well as applications in the food and beverage industry.

For example, in a medical application, an insufflation set having a luer connector may be used to connect an insufflator to a cannula which, in turn, is attached to a patient. The insufflation set may include a gas filter connected to a length of tubing. One or more luer connectors on the gas filter and/or the tubing connect the insufflation set between the insufflator and the cannula. A flow of gas, such as CO₂, may then be provided by the insufflator through the insufflation set and the cannula to the patient, the gas flowing through the gas filter of the insufflation set en route to the patent.

In many of these applications, the resistance to fluid flow can be undesirably high. For example, the resistance to gas flow through conventional insufflation sets can be large enough to significantly reduce the amount of gas that an insufflator can deliver to a patient.

### SUMMARY OF THE INVENTION

In the course of invention, it was discovered that the resistance to flow can be significantly reduced by improving conventional luer connectors. Conventional luer connectors may include mateable male and female fittings. For example, ISO International Standard 594 (hereinafter ISO 594), e.g., 594-1 dated 1986 and 594-2 dated 1998, specify a luer connector which includes a male conical fitting and a female conical fitting. (ISO International Standard 594 is incorporated herein by reference.) When the male and female fittings are coupled together, they define a passage for the flow of fluid through the luer connector. This passage has a minimum or effective inner diameter which forms a flow restriction. According to the invention the size of the flow restriction may be enlarged in the male fitting, the female fitting, or both the male and female fittings, thereby reducing the resistance to fluid flow through the luer connector.

In accordance with one aspect, the invention provides a luer connector comprising a female fitting and a male fitting. The female fitting has an open end including a bore and an opening at the end of the bore which receives the male fitting. The bore has a taper and the opening has a diameter (e.g., “D” in ISO 594) which are specified in accordance with ISO 594. The male fitting includes a projection which fits into the bore of the female fitting. The male fitting also includes a fluid passage which extends through the projection and has an effective inner diameter of at least about 0.130 inch.

In accordance with another aspect, the invention provides a male fitting of a luer connector. The male fitting is capable of being coupled to a female fitting having an open end which includes a bore having a taper and an opening having a diameter (e.g., “D” in ISO 594) as specified in accordance with ISO 594. The male fitting includes a projection which is capable of fitting into the bore of the female fitting and also includes a fluid passage which extends through the projection and has an effective diameter of at least about 0.130 inch.

Luer connectors or fittings embodying one or more of the aspects of the invention provide many advantages over conventional luer connectors and fittings. For example, embodiments of the invention as well as assemblies, such as insufflation sets, which incorporate embodiments of the invention, can provide a significantly reduced resistance to fluid flow. Consequently, many of the embodiments can provide improvements in fluid flow rates of up to about 10% to about 200% or more.

### BRIEF DESCRIPTION OF THE DRAWINGS

FIG. 1 is a sectional view of one example of a luer connector including a male fitting and a female fitting.

FIG. 2 is a block diagram of an insufflation set having a male fitting as shown in FIG. 1.

### DESCRIPTION OF EMBODIMENTS OF THE INVENTION

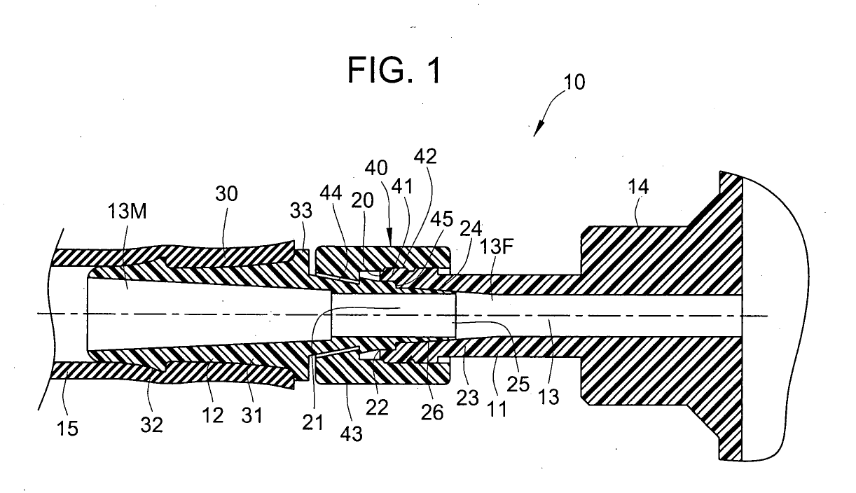

Luer connectors embodying one or more aspects of the invention may be structured in a wide variety of ways. One example of an embodiment of a luer connector 10 is shown in FIG. 1 and generally comprises a female fitting 11 which is mateable to a male fitting 12. The female and male fittings 11, 12 may be coupled to form a fluid passage 13 extending through the luer connector 10. The female and male fittings 11, 12 are preferably sealed to one another in any suitable manner to provide a reliable and sufficiently leak-free fluid connection.

A female fitting of a luer connector embodying the invention may be fabricated from various rigid or semi-rigid materials, including metallic materials or polymeric materials, and may be variously configured. Form example, the female fitting may be a separate piece formed from one or more parts and may be connected, e.g., bonded or mechanically coupled, to a structure such as a length of tubing. Alternatively, the female fitting may be formed as an integral portion of a structure, e.g., a device such as an insufflator or a filter housing. For example, the female fitting 11 shown in FIG. 1 may be an integral portion of a cannula 14.

The female fitting 11 preferably has an open end 20 which receives the male fitting 12, and the open end 20 may be capped or otherwise covered prior to insertion of the male fitting 12. The open end 20 may include a bore 21 which preferably forms a portion of the fluid passage 13F in the female fitting 11 and an opening 22 at the end of the bore 21 into which the male fitting 12 may be inserted. The bore 21 may be conical and have a taper and the opening may have a diameter (e.g., “D” in ISO 594), for example, as specified in accordance with ISO 594. For example, the taper may be about 6% and the diameter D may be between about 0.168 inch (4.270 mm) and about 0.170 inch (4.315 mm). The female fitting 11 may have one or more of the other specifications set forth in ISO International Standard 594. For example, the minimum depth of the tapered bore 21 may be about 0.295 inch (7.500 mm) and the maximum radius of curvature at the opening 22 may be about 0.02 inch (0.5 mm).

The female fitting 11 may include one or more additional features. For example, the female fitting 11 may include a body having a wall which defines the fluid passage. Although the body may be variously configured, in FIG. 1, the body 23 has a hollow, generally cylindrical configuration which preferably forms an integral extension of a cannula 14 or any other suitable structure, such as a device housing. If the female fitting is a separate piece, the body may further include an arrangement, such as a hose barb, for connecting the female fitting to a length of tubing or hose. Further the female fitting may include a cap or cover which may be removably mounted to the body to enclose the open end prior to being coupled to the male fitting.

A male fitting of a luer connector embodying the invention also may be fabricated from various rigid or semi-rigid materials, including metallic materials or polymeric materials, and may be variously configured. For example, the male fitting may be formed as an integral portion of a structure. Alternatively, the male fitting may be a separate piece formed from one or more parts and may be connected, e.g., bonded or mechanically coupled, to a structure such as a length of tubing. In the embodiment illustrated in FIG. 1, the male fitting 12 preferably is a single piece connected to tubing 15.

The male fitting 12 preferably includes a projection 24 having an open end 25, and the fluid passage 13M in the male fitting 12 extends through the projection 24 to the open end 25, the projection 24 including a wall 26 surrounding the fluid passage 13M. The projection 24 is preferably configured to fit closely within the opening and bore of a female fitting, where the opening and bore are as specified in ISO 594. For example, the exterior of the projection 24 of the male fitting 12 may be conical and have a taper substantially equal to the taper of the female fitting, e.g., about 6%, as specified in accordance with ISO 594, and the outer surface of the projection 24 is preferably dimensioned to provide contact, e.g., sealing contact, with the inner surface of the wall defining the bore 21 of the female fitting 11. The male fitting 12 is thus preferably compatible with a conventional ISO 594 female fitting. However, regardless of the configuration of the projection 24, the fluid passage 13M through the male fitting 12, including the projection 24, preferably has an effective inner diameter of at least about 0.130 inch. For example, the effective inner diameter may be at least about 0.135 inch, 0.140 inch, 0.145 inch, or 0.150 inch. In many preferred embodiments, the effective inner diameter is in the range from about 0.140 inch to about 0.150 inch, e.g., about 0.143 inch +/− about 0.003 inch.

The outer diameter of the projection 24 at the open end 25 is preferably small enough to fit within the bore 21 of the female fitting 11. For example, the outer diameter of the projection 24 at the open end 25 may be less than about 0.170 inch. Further, the outer diameter may be greater than about 0.154 inch. For many preferred embodiments, the outer diameter of the projection 24 at the open end 25 is in the range from about 0.159 inch to about 0.168 inch.

The male fitting may be formed in a variety of ways to provide a fluid passage having an effective inner diameter of at least about 0.130 inch. For example, the length of the projection from the open end may be relatively long, e.g., as long as, or longer than, the distance “E” (e.g., 0.295 inch) specified in ISO International Standard 594. However, if the effective inner diameter of the fluid passage through the projection is at least about 0.130 inch and, more preferably, about 0.145 inch, the wall of a long projection at the open end can become very thin. Consequently, a male fitting with a long projection is less preferred.

Another of the many ways to provide a fluid passage having an effective inner diameter of at least about 0.130 inch is to form a projection 24 having a length from the open end 25 of less than the distance “E” (e.g., 0.295 inch) specified in ISO International Standard 594. For example, the length of the projection 24 from the open end 25 may be less than about 0.275 inch, 0.250 inch, 0.225 inch, 0.210 inch, 0.200 inch, 0.190 inch, 0.180 inch or 0.170 inch. In may preferred embodiments, the length of the projection 24 from the open end 25 is in the range from about 0.175 inch to 0.200, e.g., about 0.185 inch +/−0.005 inch. At these lengths, the projection 24 is preferably long enough to provide sufficient contact, e.g., sealing contact, between the outer surface of the wall 26 of the projection 24 of the male fitting 12 and the inner surface of the wall forming the bore 21 of the female fitting 11. In addition, the projection 25 is preferably short enough to maintain a significant wall thickness at the open end 25 of the projection 24, for example, sufficient thickness to prevent the wall from collapsing or crimping at the end of the projection. Consequently, a male fitting 12 with a short projection is more preferred.

The male fitting 12 may include one or more additional features. For example, the male fitting 12 may include a body 30, and the body 30 may be configured in a wide variety of ways. For example, the body 30 may include a wall 31 which defines the fluid passage 13M. The body 30 preferably includes the projection 24, and a portion of body wall 31 may comprise the wall 26 of the projection 24. In the illustrated embodiment, the fluid passage 13M extends generally axially through the body 30, forming an opening on each end of the body 30, and the body wall 31 preferably defines a substantially smooth tapered passage with few, if any steps, to minimize fluid turbulence. However, the fluid passage may extend in any desirable direction through the body and may have any desired configuration within the body. The male fitting may also comprise an arrangement, such as a hose barb 32, for securing the tubing 15 to the body 30, as well as a stop 33 for limiting the distance that the tubing end of the body 30 may be inserted into the tubing 15.

In addition to the female and male fittings, the luer connector preferably includes a mechanism for interlocking the female and male fittings. The interlock mechanism is preferably a mechanical coupling, such as a ratchet mechanism or interlocking nested cylinders which are keyed to one another. In the embodiment shown in FIG. 1, the mechanical coupling 40 preferably comprises a jackscrew arrangement. For example, one of the male and female fittings, e.g., the female fitting 11, may have external threads 41, for example, at the open end 20 of the body 23, while the other fitting, e.g., the male fitting 12, may have internal threads 42 mateable with the external threads 41. The internal threads 42 are preferably disposed on a collar 43 which is rotatably mounted in a race 44 on the male fitting 12, fixing the axial position of the collar 43 on the male fitting 12.

To connect the female and male fittings 11, 12, any caps or covers protecting the projection 24 and the bore 21 may be removed; the projection 24 may be aligned with and/or inserted into the bore 21; and the internal and external threads 41, 42 may be engaged. Rotating the collar 43 in one direction within the race 44 jacks the projection 24 into engagement with the bore 21, connecting the female and male fittings 11, 12 and preferably contacting, e.g., sealingly contacting, the outer surface of the projection 24 and the inner surface of the wall forming the bore 21. Stops 45 may be provided to prevent over insertion of the projection 24 into the bore 21. Rotating the collar 43 in the opposite direction disconnects the female and male fittings 11, 12.

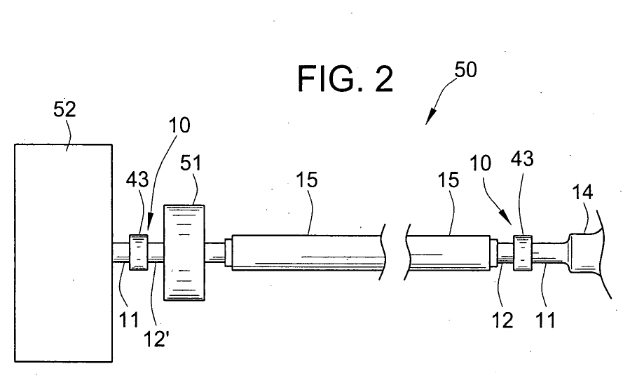

The connection provided by luer connectors and fittings embodying the invention provide many advantages over the prior art. For example, embodiments of the invention, such as the insufflation set 50 shown in FIG. 2, can provide a significantly reduced resistance to fluid flow. The insufflation set includes an inlet end and an outlet end and defines a gas flow path between the inlet end and the outlet end. In the illustrated embodiment, the insufflation set 50 preferably includes a gas filter 51 having a male fitting 12′ of a luer connector at the inlet end. The male fitting 12′ may be similar to that shown in FIG. 1 but integrally formed with the filter 51 at the inlet of the filter housing. The inflation set 50 may also include a length of tubing 15 which is attached at one end to the outlet of the filter housing and a male fitting 12 of a luer connector which may be identical to that shown in FIG. 1 and attached to the other end of the tubing 15 at the outlet end of the insufflation set 50. The gas flow path then extends between the male fittings 12′, 12 through the gas filter 51 and the tubing 15. The insufflation set 50 is preferably coupled between an insufflator 52 and a cannula 14 attached to a patient. Each of the insufflator 52 and the cannula 14 preferably include a female fitting 11 of a luer connector which may be identical or similar to that shown in FIG. 1. While the illustrated insufflation set 50 preferably has male fittings 12′, 12 on each end of the set and female fittings 11 on the insufflator 52 and the cannula 14, other variations are within the scope of the invention. For example, one or both of the male fittings and the corresponding female fittings may be exchanged such that the female fitting is on the insufflation set and the male fitting is on the insufflator or the cannula. As another example, the insufflation set may include a luer connection embodying the invention on only one end of the set and an entirely different kind of connector on the other end of the insufflation set.

Once the insufflation set 50 is coupled between the insufflator 52 and the cannula 14, a gas such as CO₂ may be directed to the patient by the insufflator 52. The insufflator 52 may be a high-flow machine capable of directing, for example, 30-40 LPM to the patient or a lower-flow machine capable of directing, for example, 10-15 LPM to the patient. Comparative testing of insufflation sets incorporating luer connectors and fittings embodying the invention and having an effective inner diameter of at least 0.130 inch with identical insufflation sets incorporating conventional ISO International Standard 594 luer connectors shows improvements in the flow rate to the patient of up to about 10% to 200% or more.

1. A luer connector comprising:
a female fitting having an open end including a bore and an opening at the end of the bore, wherein the bore has a taper and the opening has a diameter D as specified in ISO International Standard 594; and
a male fitting having a projection which fits within the bore of the female fitting, the male fitting further including a fluid passage which extends through the projection and has an effective inner diameter of at least about 0.130 inch.

2. A male fitting of a luer connector which is capable of being coupled to a female fitting having an open end which includes a bore having a taper and an opening having a diameter D as specified in ISO International Standard 594, the male fitting comprising:
a projection which is capable of fitting within the tapered bore of the female fitting and
a fluid passage which extends through the projection and has an effective diameter of at least about 0.130 inch.

3. The luer connector or male fitting of claim 1 wherein the male fitting comprises a body which includes the projection, the fluid passage extending through the body.

4. The luer connector or male fitting of claim 3 further comprising an arrangement which secures tubing to the male fitting.

5. The luer connector or male fitting of claim 1 further comprising an interlock mechanism which interlocks the male fitting and the female fitting.

6. The luer connector or male fitting of claim 5 wherein the interlock mechanism includes a mechanical coupling.

7. The luer connector or male fitting of claim 1 wherein the projection of the male fitting has a taper which is substantially equal to the taper in the bore of the female fitting.

8. The luer connector or male fitting of claim 1 wherein the length of the projection from the open end is less than about 0.295 inch.

9. The luer connector or male fitting of claim 8 wherein the length of the projection is in the range from about 0.180 inch to about 0.190 inch.

10. The luer connector or male fitting of claim 1 wherein the projection has an outer diameter at the open end which is less than about 0.170 inch.

11. The luer connector or male fitting of claim 1 wherein the effective inner diameter is greater than about 0.140 inches.

12. The luer connector or male fitting of claim 11 wherein the effective inner diameter is about 0.143 inch +/−0.003 inch.

13. An insufflation set comprising an inlet end, an outlet end, a gas flow path between the inlet end and the outlet end, a filter disposed in the gas flow path, and a luer connector or a male fitting as claimed in claim 1 connected in the gas flow path.
